(19) United States
(12) Patent Application Publication
(10) Pub. No.: US 2017/0168688 A1
(43) Pub. Date: Jun. 15, 2017

(54) Configuring a User Interface of a Dialysis Machine

(71) Applicant: Fresenius Medical Care Holdings, Inc., Waltham, MA (US)

(72) Inventor: David Yuds, Antioch, CA (US)

(21) Appl. No.: 14/963,704

(22) Filed: Dec. 9, 2015

(51) Int. Cl. G06F 3/0484; A61M 1/14; G06F 19/00; G06K 9/00; G06F 9/44; A61M 1/28; A61M 5/142

(52) U.S. Cl. CPC G06F 3/04847; A61M 1/28; A61M 1/14; A61M 5/142; G06K 9/00288; G06F 9/4443; G06F 19/3406; A61M 2205/502; A61M 2205/3584

(57) ABSTRACT

In one aspect, a dialysis system includes one or more tubes for transporting fluid to and from a dialysis patient, a display and one or more processors. The one or more processors are configured to determine an identity of a user of the dialysis system. Based on the determined identity of the user, the one or more processors access a user interface configuration profile associated with the user, and cause a user interface to appear on the display. The user interface includes one or more controls that, when invoked, cause the dialysis system to carry out a dialysis operation. The user interface is caused to appear on the display based at least in part on the identity of the user.

### TECHNICAL FIELD

This description relates to dialysis machines, and more specifically configuring dialysis machine user interfaces.

### BACKGROUND

Renal dysfunction or failure and, in particular, end-stage renal disease, causes the body to lose the ability to remove water and minerals and excrete harmful metabolites, maintain acid-base balance and control electrolyte and mineral concentrations within physiological ranges. Toxic uremic waste metabolites, including urea, creatinine, and uric acid, accumulate in the body's tissues which can result in a person's death if the filtration function of the kidney is not replaced.

Dialysis is commonly used to replace kidney function by removing these waste toxins and excess water. In one type of dialysis treatment—hemodialysis (HD)—toxins are filtered from a patient's blood externally in a hemodialysis machine. Blood passes from the patient through a dialyzer separated by a semi-permeable membrane from a large volume of externally-supplied dialysis solution. The waste and toxins dialyze out of the blood through the semi-permeable membrane into the dialysis solution, which is then typically discarded.

The dialysis solutions or dialysates used during hemodialysis typically contain sodium chloride and other electrolytes, such as calcium chloride or potassium chloride, a buffer substance, such as bicarbonate or acetate, and acid to establish a physiological pH, plus, optionally, glucose or another osmotic agent.

Another type of dialysis treatment is peritoneal dialysis (PD) that utilizes the patient's own peritoneum, a membranous lining of the abdominal body cavity. With its good perfusion properties, the peritoneum is capable of acting as a natural semi-permeable membrane for transferring water and waste products to a type of dialysate solution known as PD solution introduced temporarily into the patient's abdominal cavity. An access port is implanted in the patient's abdomen and the PD solution is infused usually by a pump into the patient's abdomen through a patient line and left to dwell for a period of time and then drained out. This procedure is usually repeated multiple times for a complete treatment. PD machines, such as automated PD (APD) machines or continuous ambulatory PD (CAPD) machines, are designed to facilitate or control the PD process so that it can be performed at home without clinical staff in attendance.

Dialysis machines are typically equipped with interfaces for receiving inputs and providing information to users.

### SUMMARY

In one aspect, a dialysis system includes one or more tubes for transporting fluid to and from a dialysis patient, a display and one or more processors. The one or more processors are configured to determine an identity of a user of the dialysis system. Based on the determined identity of the user, the one or more processors access a user interface configuration profile associated with the user, and cause a user interface to appear on the display. The user interface includes one or more controls that, when invoked, cause the dialysis system to carry out a dialysis operation. The user interface is caused to appear on the display based at least in part on the identity of the user.

In another aspect, a method includes determining an identity of a user of a dialysis system and based on the determined identity of the user, accessing a user interface configuration profile associated with the user. The method also includes causing a user interface to appear on the display, the user interface comprising one or more controls that, when invoked, cause the dialysis system to carry out a dialysis operation. The user interface is based at least in part on the identity of the user.

Implementations can include one or more of the following features.

In some implementations, a dialysis system includes one or more sensors configured to communicate with the one or more processors, the one or more sensors configured to detect identity information that can be used to identify the user.

In certain implementations, the identity information is detected using one or more of facial recognition, data transmitted according to an RFID protocol, and data transmitted according to a Bluetooth protocol.

In some implementations, the one or more processors are configured to receive, from the sensors, first data representing one or more facial features of the user; receive, from data storage, second data representing facial features of one or more authorized users of the dialysis system; determine, based on the received first and second data, that the user is an authorized user of the dialysis system; and communicate, to a database system of user interface configuration profiles associated with authorized users, a request for a user interface configuration profile associated with the authorized user. The user interface is caused to appear on the display in a configuration defined by the user interface configuration profile.

In certain implementations, the user interface is caused to appear on the display is based on an alarm triggered at the dialysis system.

In some implementations, the user interface comprises one or more operating parameters of the dialysis system.

In certain implementations, the one or more processors are configured to communicate the operating parameters of the dialysis system to a device external to the dialysis system.

In some implementations, the one or more processors are configured to communicate data representing the identity of the user to a device external to the dialysis system.

In some implementations, the one or more processors are configured to determine an identity of an administrator of the dialysis machine, and communicating the data representing the identity of the user for receipt by the administrator.

In some implementations, the dialysis system comprises a peritoneal dialysis (PD) machine, and the fluid comprises dialysate.

In certain implementations, the dialysis system comprises a hemodialysis (HD) machine, and the fluid comprises blood.

### DESCRIPTION OF DRAWINGS

FIG. 1 shows a perspective view of a hemodialysis system.

FIG. 2 shows a perspective view of a peritoneal dialysis system.

FIG. 3 shows a facility comprising plurality of dialysis systems.

FIG. 4A shows a standard user interface that appears on the display of a dialysis system.

FIG. 4B shows a user configured user interface that appears on the display of a dialysis system.

FIG. 5A shows a standard user interface that appears on the display of a dialysis system during an alarm.

FIG. 5B shows a user configured user interface that appears on the display of a dialysis system during an alarm.

FIG. 6 is a flowchart of a process of configuring the display of the user interface

FIG. 7 shows an example of a computer system.

### DETAILED DESCRIPTION

A dialysis machine can be configured to automatically detect a user and configure a user interface in a manner specific to that user. In this way, the ability of a user to attend to the dialysis treatment or react to alarms can be improved.

Modern dialysis systems often have a computerized user interface displayed on a display screen. The user interface can vary depending on how the user interface is configured, the manufacturer and the model, etc. Sometimes a user may need to reconfigure the user interface of each time he/she uses a dialysis system. For example, in a dialysis clinic context, the user may be a technician or nurse who works in a facility with many dialysis systems. In a home context, the user may be a dialysis patient or a caregiver of the patient.

However, if the dialysis system can detect the user—for example, when the user is in proximity to the system—the system can automatically change the display to a display configuration expected by the user. As a result, a user can seamlessly use any dialysis system in a facility without having to reconfigure the display. This allows the user to work with a familiar display configuration, which may be especially important if the user is responding to a medical alarm or time is otherwise of the essence. Moreover, only the desired operating parameters and controls are presented to the user which prevents the display from appearing cluttered or confusing the user with information he/she cannot use. The dialysis system can also be configured to record the identity of the user, the time and duration of system use, and the operational parameters of the dialysis system. If an unknown user tries to use the dialysis system, a supervisor can be notified.

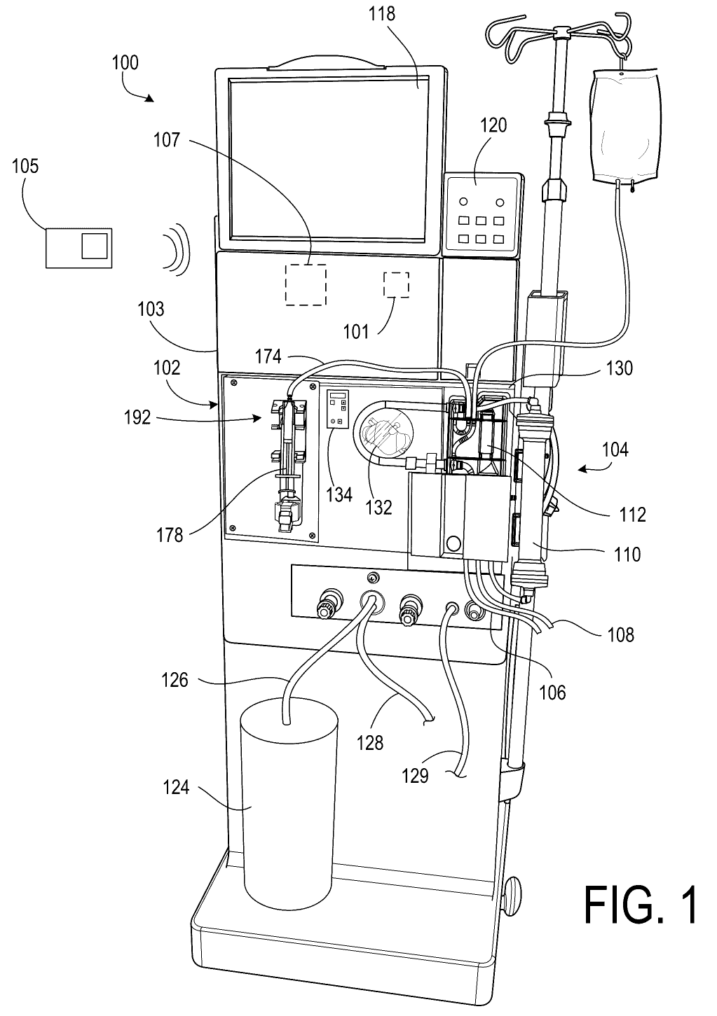

FIG. 1 shows a perspective view of a hemodialysis system 100 configured to detect the identity a user by wirelessly communicating with short-range wireless devices, such as an identification (ID) card 105 that the user may be carrying. Based on the identity of the user, a user interface appears on the display 118. The user interface, for example, can be based on data, that is associated with the user, and stored in a database of user interface configuration profile.

The hemodialysis system 100 includes a hemodialysis machine 102 connected to a disposable blood component set 104 that partially forms a blood circuit. During hemodialysis treatment, an operator connects arterial and venous patient lines 106, 108 of the blood component set 104 to a patient. The blood component set 104 includes an air release device 112, which contains a self-sealing vent assembly that allows air but does not allow liquid to pass. As a result, if blood passing through the blood circuit during treatment contains air, the air release device 112 will vent the air to atmosphere.

The blood component set 104 is secured to a module 130 attached to the front of the hemodialysis machine 102. The module 130 includes the blood pump 132 capable of circulating blood through the blood circuit. The module 130 also includes various other instruments capable of monitoring the blood flowing through the blood circuit. The module 130 includes a door that when closed, as shown in FIG. 1, cooperates with the front face of the module 130 to form a compartment sized and shaped to receive the blood component set 104. In the closed position, the door presses certain blood components of the blood component set 104 against corresponding instruments exposed on the front face of the module 130.

The operator uses a blood pump module 134 to operate the blood pump 132. The blood pump module 134 may include a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in an arterial drip chamber. Or these controls may exist in part or wholly within the graphical user interface on the touch screen accessible display 118.

The hemodialysis machine 102 further includes a dialysate circuit formed by the dialyzer 110, various other dialysate components, and dialysate lines connected to the hemodialysis machine 102. Many of these dialysate components and dialysate lines are inside the housing 103 of the hemodialysis machine 102 and are thus not visible in FIG. 1. During treatment, while the blood pump 132 circulates blood through the blood circuit, dialysate pumps (not shown) circulate dialysate through the dialysate circuit.

A dialysate container 124 is connected to the hemodialysis machine 102 via a dialysate supply line 126. A drain line 128 and an ultrafiltration line 129 also extend from the hemodialysis machine 102. The dialysate supply line 126, the drain line 128, and the ultrafiltration line 129 are fluidly connected to the various dialysate components and dialysate lines inside the housing 103 of the hemodialysis machine 102 that form part of the dialysate circuit. During hemodialysis, the dialysate supply line 126 carries fresh dialysate from the dialysate container 124 to the portion of the dialysate circuit located inside the hemodialysis machine 102. As noted above, the fresh dialysate is circulated through various dialysate lines and dialysate components, including the dialyzer 110, that form the dialysate circuit. As will be described below, as the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate (described below) and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

A drug pump 192 also extends from the front of the hemodialysis machine 102. The drug pump 192 is a syringe pump that includes a clamping mechanism configured to retain a syringe 178 of the blood component set 104. The drug pump 192 also includes a stepper motor configured to move the plunger of the syringe 178 along the axis of the syringe 178. A shaft of the stepper motor is secured to the plunger in a manner such that when the stepper motor is operated in a first direction, the shaft forces the plunger into the syringe, and when operated in a second direction, the shaft pulls the plunger out of the syringe 178. The drug pump 192 can thus be used to inject a liquid drug (e.g., heparin) from the syringe 178 into the blood circuit via a drug delivery line 174 during use, or to draw liquid from the blood circuit into the syringe 178 via the drug delivery line 174 during use.

The hemodialysis machine 102 includes a control unit 101 (e.g., a processor) configured to receive signals from and transmit signals to the display 118, the control panel 120, and a communication module 107 (e.g., a near field communication (NFC) transceiver, a sensor or a camera). The control unit 101 can also communicate with a server (e.g., an Internet server), another dialysis system, or another network resource. In various examples, this communication with the server can be via wireless communication over a telecommunications network, can be via a wired Ethernet connection to the server, and/or can be via physical transfer of a computer readable medium between the server and the dialysis system 100, such as using a USB drive. The control unit 101 controls the operating parameters of the hemodialysis machine 102 based at least in part on the signals received by the display 118, the control panel 120, and the communication module 107.

The display 118 presents a user interface to the user that can include vital signs of a patient, operational parameters of the dialysis treatment, and controls associated with the hemodialysis process. For example, the operational parameters can include ultrafiltration parameters, blood pump rate and information associated with the dialysate, hematocrit alert levels, blood pressure alarm limits, medicine infusion parameters, etc. The display 118 can include a touch screen through which the user can interact and control the hemodialysis machine 102. For example, the user can input various treatment parameters associated with the hemodialysis process.

The communication module 107 is configured to detect and communicate with the short-range wireless device—for example, an ID card 105—when the device is within its wireless communication range. The communication between the communication module 107 and the ID card 105 is facilitated by a short-range wireless technology protocol, for example, a Bluetooth protocol or an RFID protocol, such as an NFC protocol. Because the ID card 105 is associated with a user of the dialysis machine 102, the communication module 107 detects information associated with the identity of the user. The communication module 107 communicates information associated with the user to the control unit 101. In response, the control unit 101 can cause the hemodialysis machine 102 to perform an action, as described in more detail below. Similarly, when the ID card 105 is taken out of wireless communication range of the communication module 107 (e.g., the ID card 105 goes from being in wireless communication range of the communication module 107 to not being in wireless communication range of the communication module 107), the communication module 107 can send a signal to the control unit 101 indicating that the user is not present. In response, the control unit 101 can cause the hemodialysis machine 102 to perform an action.

The communication module 107 can also detect information associated with the identity of the user through a biometric authentication process that can include, for example, facial recognition, palm/finger print and iris recognition. The communication module can include a sensor (for example, a camera) that can take an image of the face, finger/palm or eye of the user, and use information associated with the image to identify the user. This can be achieved by comparing the information associated with the image with user profile information in a database.

The control unit 101 receives information associated with the identity of the user from the communication module 107 and compares it with the user interface configuration profiles for multiple users stored in a database. The database can be stored on a storage device associated with the dialysis system 100 or located at an external storage device (for example a server or a storage device associated with a different dialysis system.) Based on the comparison, the control unit 101 can retrieve the user interface configuration profile of the user. The user interface that appears on the display is based on the retrieved user interface configuration profile. For example, the operating parameters and the dialysis controls that constitute the user interface are determined from the retrieved user interface configuration profile. Additionally, identity of the user, and the identity of the supervisor of the user can also be displayed.

The control unit 101 can communicate information associated with the identity of the user or the operational parameters of the dialysis machine 102, or both, to a storage device or a mobile device. For example, the control unit 101 can communicate the identity of the user to a supervisor. The control unit 101 can identify the supervisor from the user interface configuration profile associated with the user. The communication, to the supervisor, can be in the form of a text message, email, social media type posting, or voicemail to a mobile device associated with the supervisor. Alternatively, or additionally, information associated with the identity of the user can be stored in a database. Storing or communicating the identity of the user and the operational parameter can allow for tracking of the dialysis process. For example, if a user does not correctly perform the dialysis procedure, a supervisor can intervene. In another example, if an unauthorized user tries to use the system, the supervisor can undo the changes made by the unauthorized user.

The control unit 101 can be configured to add or modify a user interface configuration profile. A user can register a short-range wireless device, for example an ID card 105, that is associated with the user, and input the desired user interface configuration profile. This can be done through a touch screen on the display 118, the control panel 120 or a kiosk that is not a part of the dialysis system 100.

The control unit 101 can be configured to detect an alarm in the hemodialysis system 100, and based on the alarm, change the user interface that appears on the display. The alarm can be caused when the value of an operational parameter satisfied a criteria. For example, an alarm can occur when the conductivity of the dialysate is above a certain value. This may require immediate attention from a nurse, and therefore the user interface that appears on the display may be changed to a form expected by and/or familiar to the nurse. This process will be discussed in detail later.

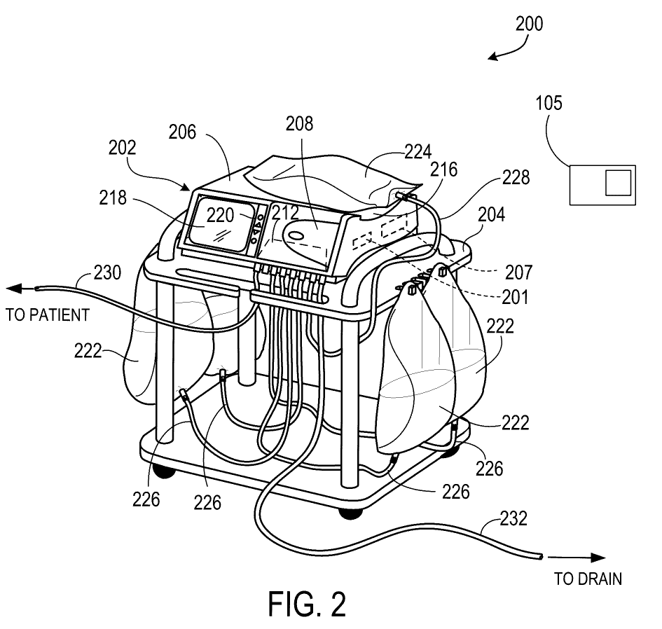

FIG. 2 shows a perspective view of a peritoneal dialysis (PD) system 200 that includes a PD cycler (also referred to as a PD machine) 202 seated on a cart 204. The PD cycler 202 includes a housing 206, a door 208, and a cassette interface (not shown) that contacts a disposable PD cassette 212 when the cassette 212 is disposed within a cassette compartment formed between the cassette interface and the closed door 208. A heater tray 216 is positioned on top of the housing 206. The heater tray 216 is sized and shaped to accommodate a bag of dialysate (e.g., a 5 liter bag of dialysate). The PD cycler 202 also includes a display 218 and additional control panel 220 that can be operated by a user (e.g., a patient) to allow, for example, set-up, initiation, and/or termination of a PD treatment.

Dialysate bags 222 are suspended from fingers on the sides of the cart 204, and a heater bag 224 is positioned in the heater tray 216. The dialysate bags 222 and the heater bag 224 are connected to the cassette 212 via dialysate bag lines 226 and a heater bag line 228, respectively. The dialysate bag lines 226 can be used to pass dialysate from dialysate bags 222 to the cassette 212 during use, and the heater bag line 228 can be used to pass dialysate back and forth between the cassette 212 and the heater bag 224 during use. In addition, a patient line 230 and a drain line 232 are connected to the cassette 212. The patient line 230 can be connected to a patient's abdomen via a catheter and can be used to pass dialysate back and forth between the cassette 212 and the patient's peritoneal cavity during use. The drain line 232 can be connected to a drain or drain receptacle and can be used to pass dialysate from the cassette 212 to the drain or drain receptacle during use.

The PD system 200, like the hemodialysis system 100, includes a control unit 201, a display 218 and a communication module 207 that can communicate with one another, and with a short-range wireless device (for example, an ID card 105). Similar to the case described in connection with the hemodialysis system 100, the PD system 200, using components such as the control unit 201 and the communication module 207, can detect information associated with the identity of a user, and change the user interface that appears on the display 218 based on the user interface configuration profile. The PD system 200 allows a user to add or modify the user interface configuration profile that can be stored in a storage device associated with the PD system, or an external storage device (for example, a server). Further, the control unit 201 of the PD system 200 can change the user interface that appears on the display based on an alarm in the PD system 200.

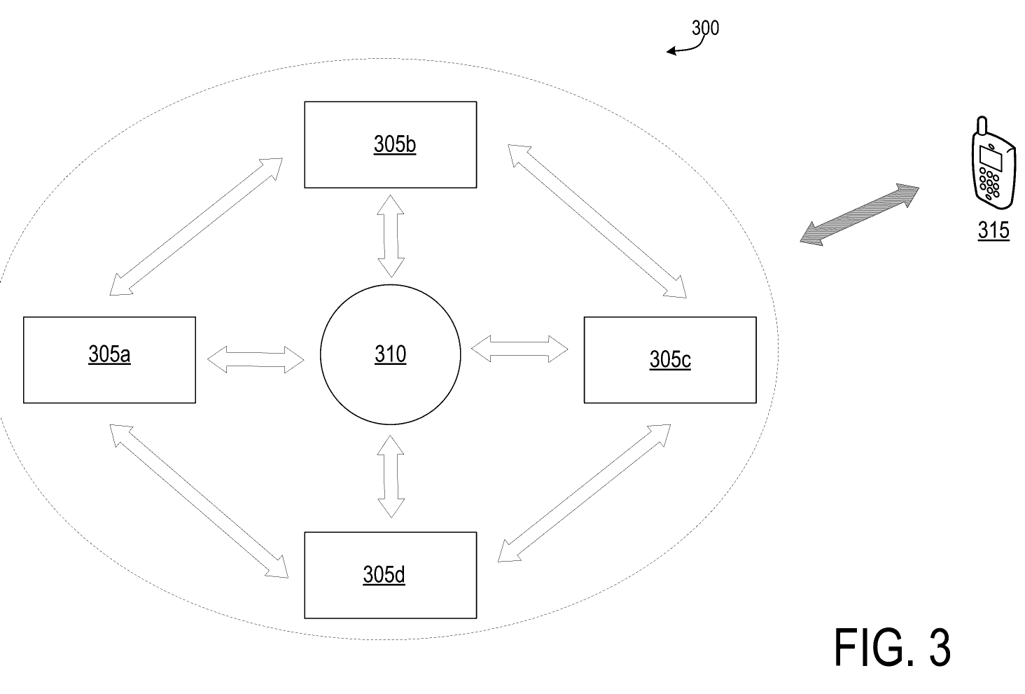

FIG. 3 shows an example of a facility 300 comprising plurality of dialysis systems 305a-d and a central server 310. The dialysis systems 305a-d can communicate with each other and with the central server 310. In an implementation, the database of user interface configuration profiles can be stored in the central server 310. When a dialysis system—for example dialysis system 305a—detects a user, it can retrieve the user interface configuration profile associated with the user from the database stored in the server. The dialysis system can also add or alter a user interface configuration profile of the database stored in the server. When a user, who has been working on a first dialysis system (for example, 305a), is detected by another dialysis system (for example, 305b), the user interface of the display of the second dialysis system will be reconfigured based on the user interface configuration profile of the user. In addition, the user may be logged out from the first dialysis machine. When a dialysis system detects a user, information associated with the identity of the user can be transmitted to an external device, for example, a cell phone 315 or the central server 310 or both. Additionally, data associated with the operational parameter and control of the dialysis may also be transmitted to an external device or the central server or both.

As mentioned before, the user interface appears on the display of the dialysis machine, and includes features such as vital signs of a patient, operational parameters of the dialysis treatment, and controls associated with the dialysis process. The user interface can include multiple sub-screens that can be accessed through tabs on the home screen. The features of the user interface can be arranged in various sub-screens in order to facilitate ease of use of the user interface. However, the manner in which the features are arranged—for example, by another user or the standard factory setting—may not be desirable to the user. Therefore, the ability to reconfigure the user interface, for example, by rearranging the features of the user interface in the various sub-screens, can increase the ease of use and efficiency of the user.

Reconfigurable user interface can also allow a user to seamlessly work on multiple dialysis machines that can have different user interfaces. Dialysis machines—made by different manufacturers, or made by the same manufacturer but having a different model—can all have different user interfaces by default. For example, the dialysis machines 305a-d at facility 300 can have different user interfaces. However, if a user can reconfigure and save his/her preferred user interface settings, and use them on multiple dialysis machines 305a-d, he/she can seamlessly move from dialysis machines to dialysis machine all while using a familiar interface.

As another example, at time of an emergency (e.g., during an alarm), a user may need to respond as soon as possible (e.g., to address a health risk to a patient). In order to expedite the response process, the user interface of the dialysis machine can be reconfigured for quick response. For example, for a given emergency, the user interface can be reconfigured to display operational parameters and controls that can be used to resolve the emergency.

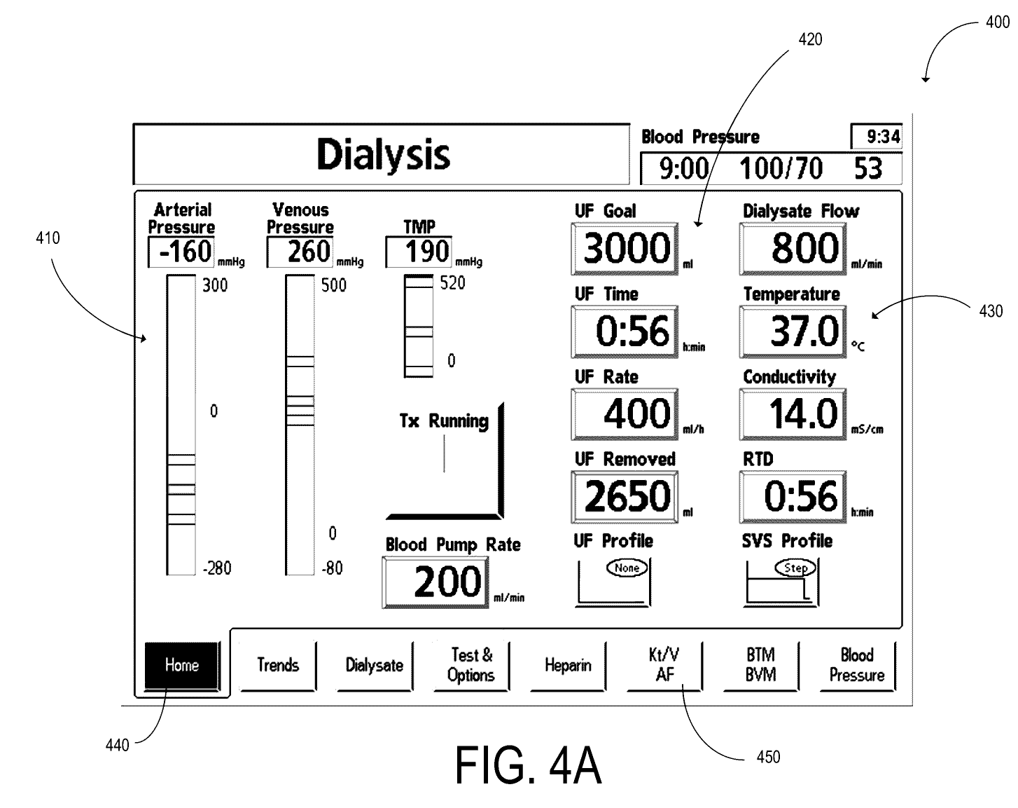

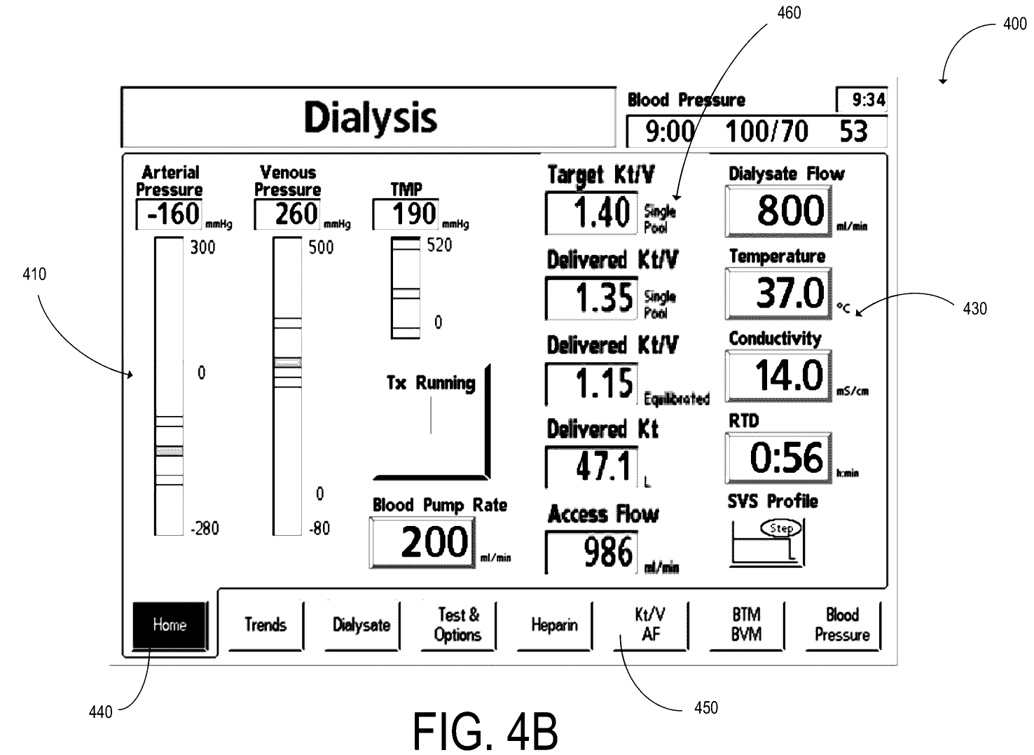

FIG. 4A shows an example of a standard user interface 400 that appears on the display of a dialysis system. The user interface includes tabs to the various sub-screens, for example, a tab to a home sub-screen 440 and a tab to a Kt/V AF sub-screen 450. The home sub-screen is usually presented when a user logs into the user interface. The home sub-screen can contain ultrafiltration features 420, features related to dialysate 430 and various pressure information associated with the dialysis process 410. The Kt/V AF sub-screen 450 includes user interface features that relate to the adequacy of the dialysis treatment. Features related to the Kt/V AF can be accessed through the Kt/V Af tab.

However, a user may desire to have features related to Kt/V AF displayed on the home sub-screen rather than in the Kt/V AF sub-screen. This may be because the user often uses Kt/V AF features, and would like to have them displayed in the home-screen for convenience. The user may be able to achieve this by reconfiguring the user interface, for example, through the touch screen of the display 118/218 or through a control panel 120/220. The user may, as described earlier, chose to save the modification in the user interface as a modification in the user interface configuration profile in a database. As a result, when the user logs into the dialysis system (or other dialysis systems in the facility that share the database of user interface configuration profiles) in the future, the home sub-screen will include features related to Kt/V AF. FIG. 4B shows the home-screen of the user interface 400 that has been reconfigured to display Kt/V AF features. For example, the ultrafiltration features 420 have been replaced by Kt/V AF features 460.

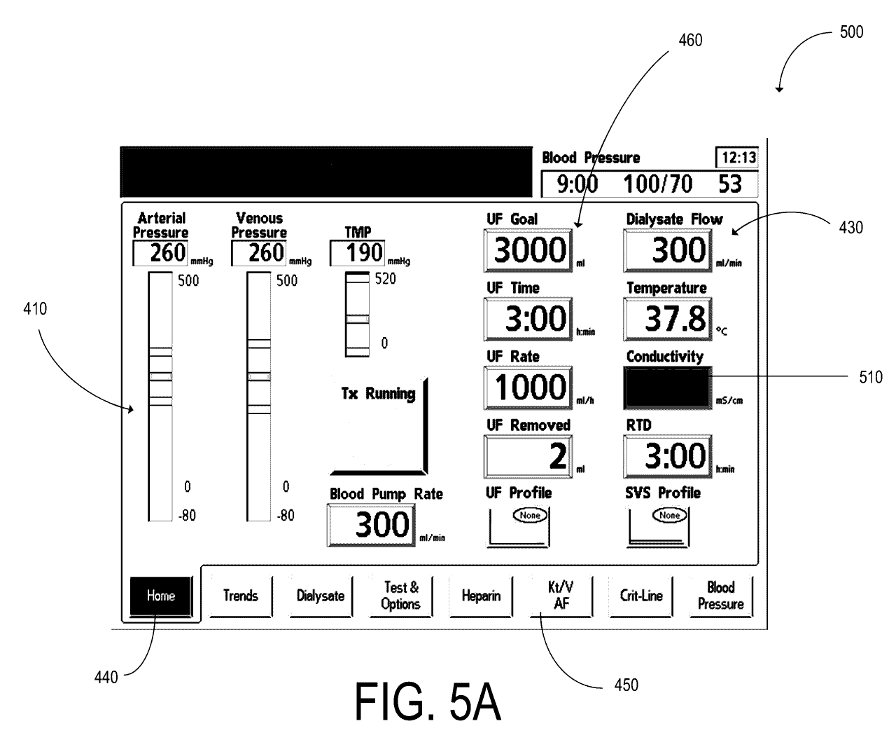

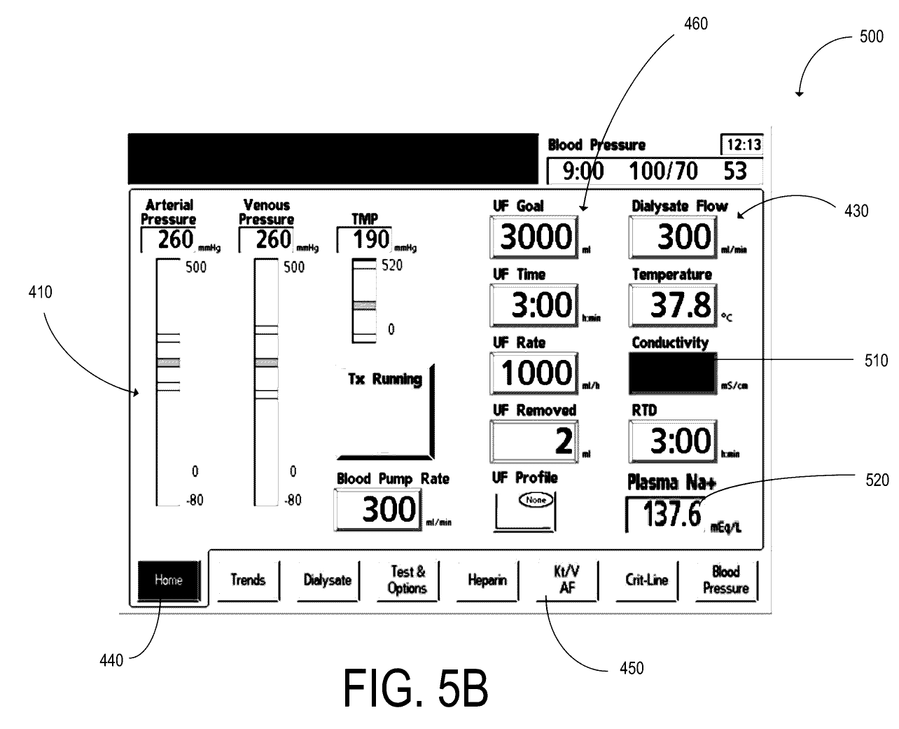

FIG. 5A shows an example of the home sub-screen of a standard user interface 500 during an alarm. In this example, the alarm is triggered by an undesired value in the conductivity 510 of the dialysate. In the event of an alarm, immediate attention from a user of the dialysis system, for example, a nurse, may be required. In order to troubleshoot the alarm, it is important for a nurse to refer to the plasma sodium value of the dialysate and the conductivity 510 of the dialysate. However, the plasma sodium value is displayed in the Kt/V AF sub-screen. Therefore, it can be challenging for the nurse to troubleshoot the alarm from the home screen, e.g., without taking valuable time to enter a different screen.

FIG. 5B shows an example of the home sub-screen of the user interface 500 that has been reconfigured by a user (e.g., the nurse) to display the plasma sodium value 520 along with the value of conductivity 510 of the dialysate. Therefore, the user can refer to both the plasma sodium value 520 and value of the conductivity 510 simultaneously. Because this reconfiguration is associated with the user, and made every time the user is using this particular dialysis machine, the ability of the user to react to the alarm is improved.

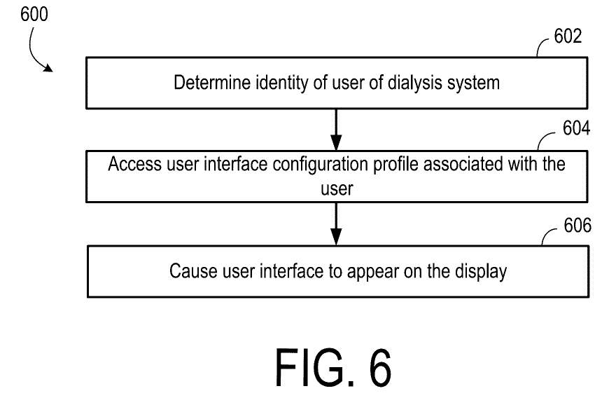

FIG. 6 illustrates a flowchart 600 that describes the process of reconfiguring the user interface of dialysis systems described in FIGS. 1 and 2. First, the identity of a user, who comes in proximity to the dialysis system, is determined 602. The user can be identified by a communication module (e.g., the communication module 107 shown in FIG. 1 or the communication module 207 shown in FIG. 2) that communicates with a short-range wireless device that is associated with the user, and detects information associated with the identity of the user. Information related to the identity of the user can also be obtained through a biometric authentication process that can include, for example, facial recognition, palm print and iris recognition. The communication module 107/207 communicates the information associated with the identity of the user to a control unit (e.g., the control unit 101 shown in FIG. 1 or the control unit 201 shown in FIG. 2).

The control unit 101 or the control unit 201, upon receiving the information associated with the identity of the user, accesses 604 user interface configuration profile associated with the user from a database of user interface configuration profiles. The database can be stored on a storage device associated with the dialysis system 100 or the dialysis system 200 or located at an external storage device (for example a server or a storage device associated with a different dialysis system.) Based on the accessed user interface configuration profile, the control unit 101 or the control unit 201 causes 606 a user interface to appear on the display 118 or the display 218.

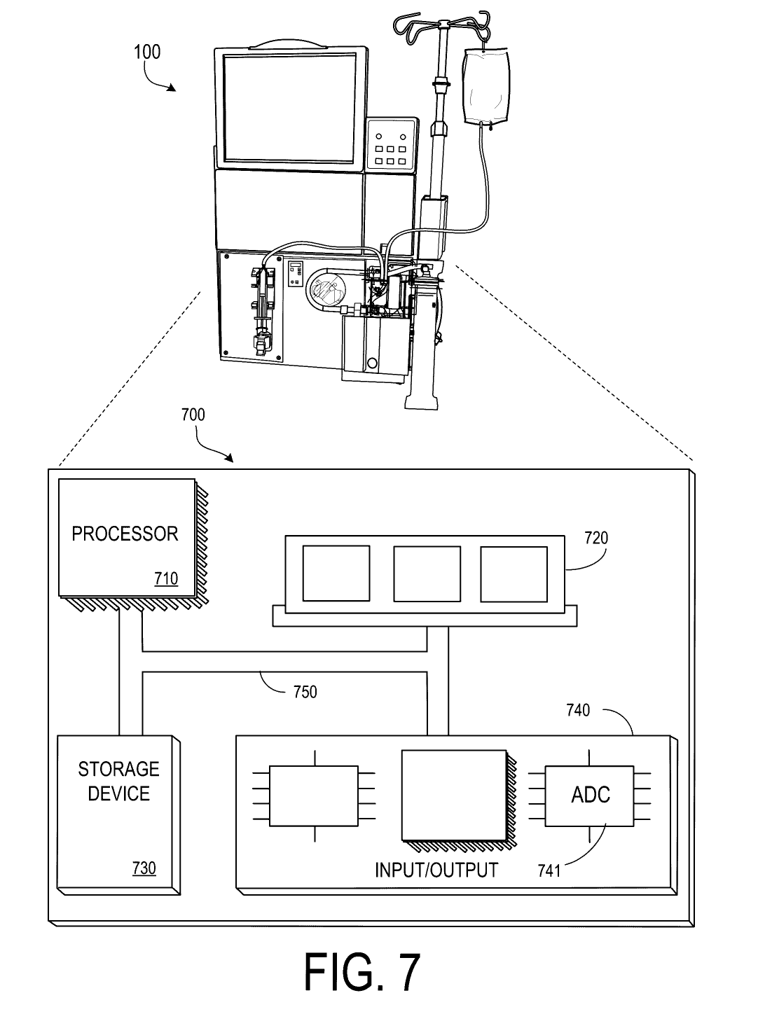

FIG. 7 is a block diagram of an example computer system 700. For example, referring to FIGS. 1 and 2, the control unit 101 or the control unit 201 could be an example of the system 700 described here. The system 700 includes a processor 710, a memory 720, a storage device 730, and an input/output interface 740. Each of the components 710, 720, 730, and 740 can be interconnected, for example, using a system bus 750. The processor 710 is capable of processing instructions for execution within the system 700. The processor 710 can be a single-threaded processor, a multi-threaded processor, or a quantum computer. The processor 710 is capable of processing instructions stored in the memory 720 or on the storage device 730. The processor 710 may execute operations such as receiving signals from a sensing element (e.g., the communication module 107 shown in FIG. 1 or the communication module 207 shown in FIG. 2) and comparing data based on the signals to stored data, e.g., data stored in a look-up table of temperature values.

The memory 720 stores information within the system 700. In some implementations, the memory 720 is a computer-readable medium. The memory 720 can, for example, be a volatile memory unit or a non-volatile memory unit.

The storage device 730 is capable of providing mass storage for the system 700. In some implementations, the storage device 730 is a non-transitory computer-readable medium. The storage device 730 can include, for example, a hard disk device, an optical disk device, a solid-date drive, a flash drive, magnetic tape, or some other large capacity storage device. The storage device 730 may alternatively be a cloud storage device, e.g., a logical storage device including multiple physical storage devices distributed on a network and accessed using a network.

The input/output interface 740 provides input/output operations for the system 700. In some implementations, the input/output interface 740 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device includes driver devices configured to receive input data and send output data to other input/output devices, e.g., keyboard, printer and display devices 118/218. In some implementations, mobile computing devices, mobile communication devices, and other devices are used.

In some implementations, the input/output interface 740 includes at least one analog-to-digital converter 741. An analog-to-digital converter converts analog signals to digital signals, e.g., digital signals suitable for processing by the processor 700. In some implementations, one or more sensing elements (e.g., the communication module 107 shown in FIG. 1 or the communication module 207 shown in FIG. 2) are in communication with the analog-to-digital converter 741.

In some implementations, the system 700 is a microcontroller. A microcontroller is a device that contains multiple elements of a computer system in a single electronics package. For example, the single electronics package could contain the processor 710, the memory 720, the storage device 730, and input/output interfaces 740.

Although an example processing system has been described in FIG. 7, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments of the invention have been described. Nevertheless, it will be understood that various modifications may be made without departing from the spirit and scope of the invention. Accordingly, other embodiments are within the scope of the following claims.

What is claimed is:

1. A dialysis system comprising:
one or more tubes for transporting fluid to and from a dialysis patient;
a display; and
one or more processors configured to
determine an identity of a user of the dialysis system,
based on the determined identity of the user, access a user interface configuration profile associated with the user, and
cause a user interface to appear on the display, the user interface comprising one or more controls that, when invoked, cause the dialysis system to carry out a dialysis operation,
wherein the user interface is caused to appear on the display based at least in part on the identity of the user.

2. The system of claim 1 comprising one or more sensors configured to communicate with the one or more processors, the one or more sensors configured to detect identity information that can be used to identify the user.

3. The system of claim 2, wherein the identity information is detected using one or more of facial recognition, data transmitted according to an RFID protocol, and data transmitted according to a Bluetooth protocol.

4. The system of claim 2, wherein the one or more processors are configured to
receive, from the sensors, first data representing one or more facial features of the user;
receive, from data storage, second data representing facial features of one or more authorized users of the dialysis system;
determine, based on the received first and second data, that the user is an authorized user of the dialysis system; and
communicate, to a database system of user interface configuration profiles associated with authorized users, a request for a user interface configuration profile associated with the authorized user;
wherein the user interface is caused to appear on the display in a configuration defined by the user interface configuration profile.

5. The system of claim 1, wherein the user interface is caused to appear on the display is based on an alarm triggered at the dialysis system.

6. The system of claim 1, wherein the user interface comprises one or more operating parameters of the dialysis system.

7. The system of claim 6, wherein the one or more processors are configured to communicate the operating parameters of the dialysis system to a device external to the dialysis system.

8. The system of claim 1, wherein the one or more processors are configured to communicate data representing the identity of the user to a device external to the dialysis system.

9. The system of claim 8, wherein the one or more processors are configured to determine an identity of an administrator of the dialysis machine, and communicating the data representing the identity of the user for receipt by the administrator.

10. The system of claim 1, wherein the dialysis system comprises a peritoneal dialysis (PD) machine, and the fluid comprises dialysate.

11. The system of claim 1, wherein the dialysis system comprises a hemodialysis (HD) machine, and the fluid comprises blood.

12. A method comprising:
determining an identity of a user of a dialysis system,
based on the determined identity of the user, accessing a user interface configuration profile associated with the user, and
causing a user interface to appear on the display, the user interface comprising one or more controls that, when invoked, cause the dialysis system to carry out a dialysis operation,
wherein, the user interface is based at least in part on the identity of the user.

13. The method of claim 12, wherein the identity is determined based on data received using one or more of facial recognition, data transmitted according to an RFID protocol, and data transmitted according to a Bluetooth protocol.

14. The method of claim 12, wherein the user interface is caused to appear on the display is based on an alarm triggered at the dialysis system.

15. The method of claim 12, wherein the user interface comprises one or more operating parameters of the dialysis system.

16. The method of claim 12, wherein the dialysis system comprises a peritoneal dialysis (PD) machine, and the fluid comprises dialysate.

17. The method of claim 12, wherein the dialysis system comprises a hemodialysis (HD) machine, and the fluid comprises blood.
